SEQUENCE LISTING (1 sequence)

SEQ ID NO: 1 - DNA, 15 bp, Artificial Sequence
  Synthetic DNA sequence that silences miRNA 22
cttcaactggcagct

(19) United States
(12) Patent Application Publication
(10) Pub. No.: US 2018/0042847 A1
(43) Pub. Date: Feb. 15, 2018

(54) METHOD OF TREATMENT COMPRISING MEMBRANE-ENCLOSED VESICLE

(71) Applicant: Duncan ROSS, Miami, FL (US)

(72) Inventor: Duncan ROSS, Miami, FL (US)

(73) Assignee: Duncan Ross, Miami, FL (US)

(21) Appl. No.: 15/557,058

(22) PCT Filed: Mar. 16, 2016
(86) PCT No.: PCT/US2016/022629

§ 371 Date: Sep. 8, 2017

Related U.S. Application Data
(60) Provisional application No. 62/133,851, filed on Mar. 16, 2015.

(51) Int. Cl. A61K 9/127; A61K 38/18; A61K 35/28

(52) U.S. Cl. CPC A61K 9/127; A61K 35/28; A61K 38/18

(57) ABSTRACT

The present disclosure relates to methods of treatment for various diseases, including chronic obstructive pulmonary disease, involving administration of compositions comprising membrane-enclosed vesicles. The membrane-enclosed vesicles may be derived from a stem cell such as a mesenchymal stem cell.

### CROSS REFERENCE TO RELATED APPLICATIONS

This application claims benefit of U.S. Provisional Application No 62/133,851, filed Mar. 16, 2015, which is hereby incorporated by reference.

### FIELD

The present disclosure relates to methods of treatment for various diseases or medical conditions such as chronic obstructive pulmonary disease or cancer involving administration of membrane-enclosed vesicles.

### BACKGROUND

Membrane-enclosed vesicles perform a variety of functions in a living organism. For example, membrane-enclosed vesicles are involved in transportation of cellular materials, enzyme storage, and metabolism. These fundamental cellular processes carried out by membrane-enclosed vesicles help maintain equilibrium of contents and activities, both within cells (intracellular) and outside of cells (extracellular). With an increasing understanding of the roles of membrane-enclosed vesicles in healthy cellular functions, opportunities to expand their application to therapeutics using healthy membrane-enclosed vesicles have become of particular interest to the inventors. For example, if a subject experiences inflammation of organs, e.g., lungs and skin, the membrane-enclosed vesicles in the healthy subject may through normal cellular function provide some ranges of anti-inflammatory response. Alternatively, administration of healthy membrane-enclosed vesicles to a subject with dysfunctional membrane-enclosed vesicles may help supplement or improve cellular functions that are associated with certain membrane-enclosed vesicles. The present disclosure is directed towards a novel method of administering membrane-enclosed vesicles to a subject in order to provide a therapeutic benefit for various diseases and disorders. Specifically, the present invention is directed towards administering membrane-enclosed vesicles to a patient to treat and or reduce the incidence of an inflammatory response, or other symptoms due to various diseases such as, for example, chronic obstructive pulmonary disease, alopecia areata, skin burns or Graft-versus-host disease.

### SUMMARY OF THE INVENTION

In one embodiment of the present disclosure, a method of treating and/or reducing conditions associated with inflammation comprises administering to a patient in need thereof a therapeutically effective amount of a membrane-enclosed vesicle from a cell. In some embodiments, the condition associated with inflammation is chronic obstructive pulmonary disease (COPD). In another embodiment, the condition associated with inflammation is induced by Graft-versus-host disease (GVHD). In another embodiment, the condition is arthritis.

In one embodiment of the present disclosure, the method of treating and/or reducing inflammation comprises administering membrane-enclosed vesicle from one or more (a mixture) of cells selected from mesenchymal stem cell, amnion-derived multipotent progenitor cell, chorion derived mesenchymal stem cell, induced pluripotent stem cell, keratinocyte, fibroblast, embryonic stem cell, ectodermal stromal cell, endodermal stromal cell, neural stem cell, lung epithelial cell, chondrocyte, hepatocyte progenitor cell, olfactory ensheathing cell, dental pulp stem cell, immortalized mesenchymal stem cell. In a certain embodiment, the membrane-enclosed vesicle is from a mesenchymal stem cell. In another embodiment, the membrane-enclosed vesicle is from a human cell.

In one embodiment of the present disclosure, the method of treating and/or reducing inflammation comprises administering membrane-enclosed vesicle, wherein the membrane-enclosed vesicle is an endosome, an exosome and/or a microvesicle. In another embodiment, the membrane of the membrane-enclosed vesicle is from the plasma membrane. In a further embodiment, the plasma membrane-enclosed vesicle is substantially free of major histocompatibility complex (MCH).

In one embodiment of the present disclosure, the method of treating and/or reducing inflammation comprises administering membrane-enclosed vesicle, wherein the membrane-enclosed vesicle is about 10 nm to about 200 nm in diameter. In another embodiment, the membrane-enclosed vesicle is about 30 nm to about 100 nm in diameter.

In one embodiment of the present disclosure, the method of treating and/or reducing inflammation comprises administering membrane-enclosed vesicle in one or more dosage forms selected from the group consisting of a solid dosage form, a cream, an aqueous mixture, a lyophilized aqueous mixture and an aerosol. In another embodiment, the membrane-enclosed vesicle is administered orally, intravenously or by inhalation. In a further embodiment, the membrane-enclosed vesicle is administered inhalationally with the use of a nebulizer.

In one embodiment of the present disclosure, the method of treating and/or reducing inflammation comprises administering a pharmaceutical composition comprising membrane-enclosed vesicles and one or more pharmaceutical acceptable carriers.

In one embodiment of the present disclosure, the method of treating and/or reducing inflammation comprises administering a pharmaceutical composition comprising membrane- enclosed vesicles and additional therapeutic agent. In another embodiment, a pharmaceutical composition comprising membrane-enclosed vesicles comprises one or more growth factors, nucleic acids or chemicals selected from the group consisting of GDF-1, FGF-1, TGF-b, TGF-b2, TGFb3, EGF, miR-133b, bFGF, TIMP1, TIMP2, TIMP3, TIMP4, Wnt4 (protein or mRNA), PDGF-AA, PDGF-BB, G-CSF, VEGF, MCP-1, MMP-1-9, PGK, IL-6, IL-7, IL-8, IL-10, IDO IL-16, BMP1, BDNF, HGF, KGF, IFN-g, E-cadherin, Fibronectin, Hsp90, gp96, Myosin, Keratin, Annexin I, Aldehyde Dehydrogenase, ATP synthase, Insulin like growth factor binding protein 1, GM-CSF, IGF like family member, miR-7, miR-13, miR-22, miR-26a, miR-27, miR-29, miR-29a, miR-30a, miR-100, miR-103, miR-106, miR-107, miR-122, miR-133, miR-140, miR-142-3p, miR-155, miR-210, miR-411, miR-483-5p, miR-502-5p, miR-FOXP3, GDNF. Hydrocortisone, Bacitracin, Neomycin Sulfate, Polymyxin B Sulfate, Pramoxine HCL, silver sulfadiazine, calendula, or a nucleic acid of sequence: +C+T+T+C+A+A+C+T+G+G+C+A+G+C+T, citric acid, sodium chloride, GSK-3787, TLR3, TLR4, quercetin, indomethacin, insulin, dexamethasone, IBMX, rosiglitazone, ascorbate-2-phosphate, selenious acid, transferrin, sodium pyruvate.

In one embodiment of the present disclosure, a method of treating a burn comprises administering to a patient in need thereof a therapeutically effective amount of a membrane-enclosed vesicle from a cell. In one embodiment of the present disclosure, the method of treating burn comprises administering membrane-enclosed vesicle from one or more cells selected from mesenchymal stem cell, amnion-derived multipotent progenitor cell, chorion derived mesenchymal stem cell, induced pluripotent stem cell, keratinocyte, fibroblast, embryonic stem cell, ectodermal stromal cell, endodermal stromal cell, olfactory ensheathing cell, dental pulp stem cell, immortalized mesenchymal stem cell. In a certain embodiment, the membrane- enclosed vesicle is from a mesenchymal stem cell. In another embodiment, the membrane-enclosed vesicle is from a human cell.

In one embodiment of the present disclosure, the method of treating burns comprises administering membrane-enclosed vesicle, wherein the membrane-enclosed vesicle is an endosome, an exosome and/or a microvesicle. In another embodiment, the membrane of the membrane-enclosed vesicle is from the plasma membrane. In a further embodiment, the plasma membrane-enclosed vesicle is substantially free of major histocompatibility complex (MCH).

In one embodiment of the present disclosure, the method of treating burn comprises administering membrane-enclosed vesicle, wherein the membrane-enclosed vesicle is about 10 nm to about 200 nm in diameter. In another embodiment, the membrane-enclosed vesicle is about 30 nm to about 100 nm in diameter.

In one embodiment of the present disclosure, the method of treating burn comprises administering membrane-enclosed vesicle in one or more dosage forms selected from the group consisting of a solid dosage form, a cream, an aqueous mixture, a lyophilized aqueous mixture and an aerosol. In another embodiment, the membrane-enclosed vesicle is administered orally, intravenously or by inhalation. In a further embodiment, the membrane-enclosed vesicle is administered inhalationally with the use of a nebulizer.

In one embodiment of the present disclosure, the method of treating and/or reducing inflammation comprises administering a pharmaceutical composition comprising membrane-enclosed vesicle and one or more pharmaceutical acceptable carriers.

In one embodiment of the present disclosure, the method of treating burn comprises administering a pharmaceutical composition comprising membrane-enclosed vesicle and additional therapeutic agent. In another embodiment, a pharmaceutical composition comprising membrane-enclosed vesicle comprises one or more growth factors selected from the group consisting of GDF-1, FGF-1, TGF-b, TGF-b2, TGFb3, EGF, miR-133b, bFGF, TIMP1, TIMP2, TIMP3, TIMP4, Wnt4 (protein or mRNA), PDGF-AA, PDGF-BB, G-CSF, VEGF, PGK, MCP-1, IL-6, IL-7, IL-8, IL-10, IDO IL-16, BMP1, BDNF, HGF, KGF, IFNg, MMP-1-9, E-cadherin, Fibronectin, Hsp90, gp96, Myosin, Keratin, Annexin I, Aldehyde Dehydrogenase, ATP synthase, Insulin like growth factor binding protein 1, GM-CSF, IGF like family member, miR-7, miR-100, miR-103, miR-106, miR-107, FOXP3, and GDNF.

In one embodiment of the present disclosure, the method of increasing the retention of fat after autologous fat transplantation comprises administering to a patient an effective amount of a membrane-enclosed vesicle from a cell.

In one embodiment of the present disclosure the method of treating solid tumors by engineering an autologous or allogeneic cell that secretes microvesicles augmented in levels of TRAIL, TNF-α, IL-1, IL-2, IL-4, VEGF, miR-122, miR-22, 483-5p, PD-1, PD-L1, IL-2, IL-6, P53, HER, neu, erbBB2, BRAF, BCR-ABL, AKT, PDK-1, PLK-1, S6K, EGFR, ALK, DHH, IHH, SHH, HR, RAD50, miRNA-22, miRNA-122, ziv-aflibercept, TLR-3, TLR-4, anti-CD20, anti-CD274, anti-CD279.

In one embodiment of the present disclosure, the method of treating or diagnosing solid tumors wherein an autologous or allogeneic tumor infiltrating mesenchymal stem cell or mesenchymal stem cell is loaded with an anti-neoplastic agent such as abiraterone acetate, gemcitabine, curcumin, bleomycin, Ceritinib cytarabine, cisplatin, taxol, docetaxel, paclitaxel, thalidomide, thiotepa. Topotecan, arsenic trioxide, bortezomib, maytansinoid DMI, letrozole, lapatanib ditosylate, exemestane, anastrozole, fulvestrant, toremifene, everolimus, sirolimus, tacrolitnus, plerixafor, gold nano-particle, rutin, cyclophosphamide, busulfan, paclitaxel, carmustine, prednisone, provenge, peg interferon, sonidegib, vismodegib, MESNA, mercaptopurine mitomycin-C, Interferon Alpha FOLFIRI, imatinib mesylate, 5-azacytydine, decitabine 2-deoxy-d-glucose. alitretinoin pazopanib hydrochloride, radium 223 dichloride, Calcium-47, Carbon-11, Carbon-14, Chromium-51 Cobalt-57 Cobalt-58, Erbium-169, Fluorine-18, Gallium-67, Gallium-68, Hydrogen-3, Indium-111 Iodine-123, Iodine-125, Iodine-131, Iron-59, Krypton-81m, 5-FU, Nitrogen-13, Oxygen-15,Phosphorus-32, Radium-223,Rubidium-82,Samarium-153,Selenium-75, Sodium-22, Sodium-24, Strontium-89, Technetium-99m, Thallium 201, Xenon-133, Yttrium-90, miR-15, let-7, miR-16, miR-mir-17-5p, miR-29, miR-34, miR-124a, miR-127, miR-143, miR-145, miR-181, miR-497, miR-31, miR-355, miR-320 miR-127 miR-30a-3p, miR-197, miR-191, miR-92a, miR-93, miR-222, miR-1826 miR-34a, miR-141a, miR-200, miR-205, miR-328, and administered to the patient for delivery to the tumor site or secretion of microvesicles.

### BRIEF DESCRIPTION OF THE DRAWINGS

FIG. 1 illustrates a patient who suffered 2nd degree burns to the majority of his face who received topical treatment of adipose derived mesenchymal stem cell membrane vesicles via medium mist spray for a period of 7 days.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

As used herein, the term “membrane-enclosed vesicle” refers to extracellular or intracellular organelle enclosed by a lipid bilayer membrane. The membrane-enclosed vesicle may be isolated from a human or non-human cell, or may be simply synthesized or manufactured. The membrane-enclosed vesicle encapsulates various bio-molecules, such as proteins, growth factors, RNA, DNA, and the like. Non-limiting examples of membrane-enclosed vesicle includes, exosome, endosome, microvesicle, liposome, lysosome, and the like.

As used herein, the term “microvesicle” refers to a type of membrane-enclosed vesicle, derived from fragments of plasma membrane.

As used herein, the term “endosome” refers to a type of intracellular membrane-enclosed vesicle involved in cellular digestion. Endosome as used herein is not limited to any one particular type of intracellular vesicle or to any one particular stage of intracellular digestion. Endosome as used herein is meant to include, but are not limited to, early endosomes, late endosomes, and recycling endosomes.

As used herein, the term “exosome” refers to a type of extracellular membrane-enclosed vesicle, which contains molecular constituents of the cell in which it was secreted from.

As used herein, the term “growth factors” refers to a peptide or protein that stimulates the growth, differentiation, proliferation, and/or healing of cells via interaction with specific cell surface receptor.

As used herein, a “subject” may be a human, non-human primate, mammal, rat, mouse, cow, horse, pig, sheep, goat, dog, cat and the like. The subject may be suspected of having or at risk for having diseases, such as inflammatory diseases and/or conditions, neurodevelopmental disorders, alcohol-induced disorders, and/or

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “pharmaceutical composition comprising optional excipient” means that the excipient may or may not be present in said pharmaceutical composition.

“Pharmaceutically acceptable carrier” or “pharmaceutically acceptable diluent” or “pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“An “effective amount” refers to a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to a castration-resistant form. Typically, a prophylactic dose is used in subjects prior to or at an earlier stage of disease, so that a prophylactically effective amount may be less than a therapeutically effective amount.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

### Membrane-Enclosed Vesicles

Membrane-enclosed vesicles are extracellular or intracellular organelles which are enclosed by a lipid bilayer membrane, containing molecular constituents of the cell in which it originated from. For example, membrane-enclosed vesicles include exosomes, endosomes, microvesicles, liposomes, lysosomes, and the like. Some membrane-enclosed vesicles are extracellular, e.g., exosome, and some membrane-enclosed vesicles are intracellular, e.g., endosome. Extracellular membrane-enclosed vesicles carry and transfer molecules and other cellular content from one cell to another by a process commonly known as membrane vesicle trafficking. This process is believed to influence many biological and cellular processes, including the immune system.

### Exosomes

Exosomes are formed when secreted by the cells in which it originated from and contains, for example, cell-specific proteins, lipids, and genetic materials. Exosomes are found in many biological fluids, including blood, urine, and cell culture medium. It is understood that exosomes play an important role in intercellular signaling and communication, coagulation, as well as waste management (Raposo, G. et al. J. Cell Biol. 2013, 200, 373-383).

Exosomes are small in size with a range of diameters between about 2 nm and about 200 nm. Exosomes may have a range of size of diameters, such as between 2 nm to 20 nm, 2 nm to 50 nm, 2 nm to 100 nm, 2 nm to 150 nm or 2 nm to 200 nm. Exosomes may have a range of size of diameters, such as between 10 nm to 20 nm, 10 nm to 50 nm, 10 nm to 100 nm, 10 nm to 150 nm or 10 nm to 200 nm. Exosome may have a range of size of diameters between 20 nm to 50 nm, 20 nm to 100 nm, 20 nm to 150 nm or 20 nm to 200 nm. Exosomes may have a range of size of diameters, such as between 30 nm to 50 nm, 30 nm to 100 nm, 30 nm to 150 nm or 30 nm to 200 nm. Exosomes may have a range of size of diameters, such as between 50 nm to 100 nm, 50 nm to 150 nm or 50 nm to 200 nm. Exosomes may have a range of size of diameters, such as between 100 nm to 150 nm or 100 nm to 200 nm. An Exosome may have a size of a diameter between 150 nm to 200 nm.

The size of an exosome may be determined by various means known in the art. For example, the size of the exosome may be determined by size fractionation and filtration through a membrane with the relevant size cut-off and determined by tracking segregation of component proteins with sodium dodecyl sulfate polyacrylamide gel electrophoresis (SDS-PAGE) or by a biological assay. Alternatively, the size may also be determined by electron microscopy.

### Preparation and Isolation of Exosomes

Exosomes may be prepared and/or isolated in a variety of ways. In one embodiment, a method involves isolating exosomes from mesenchymal stem cells (MSCs). MSCs may be prepared by an in vitro proliferation of cell culture, for example, by dispersing an embryonic stem cell colony. Other cells in which exosomes can be isolated include, but are not limited to, amnion-derived multipotent progenitor cell, chorion derived mesenchymal stem cell, induced pluripotent stem cell, keratinocyte, fibroblast, embryonic stem cell, ectodermal stromal cell, endodermal stromal cell, olfactory ensheathing cell, dental pulp stem cell, immortalized mesenchymal stem cell.

Isolation of the exosomes from MSCs may be done in a mesenchymal stem cell conditioned medium (MSC-CM). The MSC-CM may be obtained by culturing MSCs, descendent thereof or a cell line derived therefrom in a cell culture medium and isolating the cell culture medium. The MSC-CM may be filtered and/or concentrated during, prior to and/or subsequent to separation. The MSC-CM may be filtered through a membrane which has a particular porous size or a particular molecular weight cut-off. It may be subject to tangential force filtration or ultrafiltration.

Exosomes may also be synthesized or manufactured artificially, i.e., not isolated from a human or non-human cell. Instead of being isolated, exosomes could be synthesized by various lipid formation technologies.

Exosomes isolated from a human or non-human cell, or synthesized can also be modified as needed for a particular treatment and/or use. For example, biomolecules such as proteins or growth factors may be inserted (or removed) where desired. In one embodiment, 1,25-dihydroxycholecalciferol, BMP-1, Cadherin 11, KDR, Collagen Type I, Collagen Type II, Collagen Type III, Collagen Type IV, Collagen Type V, Collagen Type VI GDF-1, EGF, FGF-1, FGF-6, Osteonectin, enolase 2, enolase 1, SDF-1, CSF-1, CSF-2, CSF-3, LIF-1, b-glycerophosphate, Fibrillin 1, Fibrillin-2, HSP-70, TGF-b, TGF-b2, TGFb3, EGF, ILF, miR-133b, bFGF, TIMP1, TIMP2, TIMP3, TIMP4, Wnt4 (protein or mRNA), PDGF-AA, PDGF-BB, G-CSF, VEGF, PGK, Kit-ligand, MCP-1, IL-6, IL-7, IL-8, IL-10, IDO IL-16, BMP1, MCP-1, Rantes, BDNF, HGF, KGF, Procollagen, IFNg, MMP-1-9, E-cadherin, Fibronectin, Hsp90, gp96, Myosin, Keratin, Annexin I, Aldehyde Dehydrogenase, ATP synthase, Insulin like growth factor binding protein 1, RANKL, GM-CSF, IGF like family member, miR-7, miR-100, miR-103, miR-106, miR-107, FOXP3, Magnesium, Zinc, Boron, Iron, Fluoride, Copper, Vitamins A, K, E, D and C and/or GDNF may be included and thus encapsulated by the exosomes.

Different physical or biological properties of the exosome may be used to separate the exosome from other components of MSC or MSC-CM, for example, based on molecular weight, size, shape, composition or biological activity. For example, high performance liquid chromatography (HPLC) with various columns may be used for separation of the exosomes. The columns may be size exclusion columns or binding columns. The monitoring of the exosomes during preparation and/or separation processes in MSC-CM may be carried out using, for example, light scattering, refractive index, fluorescently labeled antibodies, dynamic light scattering or UV-visible detectors. Similarly, other types of membrane-enclosed vesicles or compositions comprising said vesicles may be prepared and/or isolated by methods described herein or by methods commonly known in the art.

### Membrane-Enclosed Vesicle Compositions for Therapeutic Use

In one embodiment, the membrane-enclosed vesicle composition may be useful for the treatment of diseases or conditions associated with inflammation. In some embodiments, the membrane-enclosed vesicle composition may be useful for reducing the incidence of inflammation, modulating, or preventing inflammation. Non limiting examples of inflammatory conditions include respiratory diseases, e.g., acute respiratory distress syndrome, chronic obstructive pulmonary disease (COPD) including asthma, chronic bronchitis, pulmonary emphysema, and silicosis, and other inflammatory conditions including burn, joint inflammation, inflammatory bowel disease, Crohn's disease, rheumatoid arthritis, rheumatoid spondylitis, osteoarthritis, ulcerative colitis, chronic glomerulonephritis, dermatitis, Multiple Sclerosis, ALS, Stroke and Graft-versus-host disease (GVHD).

In one embodiment, the membrane-enclosed vesicle composition may be useful for the treatment of osteoporosis. Osteoporosis is a degenerative disease of the bones that strikes older patients, diabetic patients and post-menopausal patients and is caused by an imbalance between bone resorption and bone formation. Mesenchymal stem cells are capable of differentiation into bone, cartilage or adipose tissue. Microvesicles secreted from MSC have been shown to contain BMP-1 RANKL, and Cadherin 11 which have been shown to stimulate bone formation. Patients with inflammatory diseases prescribed glucocorticoids often experience decreases in bone mineral density over the course of treatment. In one embodiment, the membrane-enclosed vesicle composition is useful for the treatment of inflammation as a result of Osteoporosis. In one embodiment, the membrane-enclosed vesicle composition is useful for the treatment of inflammation as a result of COPD, Osteoporosis and/or GVHD.

Non-limiting examples of osteoporosis causing disorders are Turner Syndrome, Chronic Obstructive Pulmonary Disease, hypothalamic amenorrhea, oophorectomy, ovarian failure, Cushings syndrome, Crohns disease, cystic fibrosis, Ulcerative Colitis, lactose intolerance, rheumatoid arthritis, systemic lupus, diabetes renal insufficiency, multiple myeloma, scoliosis, parkinsons disease, hypothyroidism, diabetes mellitus type I and II, acromegaly, adrenal insufficiency, andropause, post-menopausal osteoporisis, prolonged glucocorticoid, heparin, lithium or warfarin use. In one embodiment, the membrane-enclosed vesicle composition is useful for the treatment of primary osteoporosis. In one embodiment, the membrane enclosed vesicle composition is useful for the treatment of secondary osteoporosis. In one embodiment, the membrane enclosed vesicle composition is useful for the treatment of menopause in human females. In one embodiment, the membrane enclosed vesicle composition is useful for the treatment of andropause or testosterone insufficieny in human males.

In one embodiment, the membrane-enclosed vesicle composition may be useful for the treatment of neurodevelopmental disorders. Non-limiting examples of neurodevelopmental disorders include autism, autistic disorder, autistic spectrum disorder, pervasive developmental disorder, attention deficit hyperactivity disorder, DAMP (deficits in attention, motor control and perception), schizophrenia, and obsessive-compulsive disorder. For example, Autism rates have been increasing in western society and has been linked with inflammation in the mother during pregnancy potentially towards fetal brain proteins (MAR Test) or gestational flu vaccination, genetic predisposition or childhood vaccinations in the pediatric subject. Pramparo et al (archpsyc 2015) have achieved an 83% autism diagnosis accuracy as compared to classical behavior tests by quantifying increased immune/inflammatory gene expression in circulating leukocytes of toddlers. Experiments by Capecchi et al (Cell 2008) with Hoxb8 genetic knockout mice have shown a hematopoietic/immune origin to neuropsychological disorders. MSC derived microvesicles contain anti-inflammatory cytokines such as TGF-b and IL-10 which have been shown to down regulate immune responses and cells of the monocyte lineage. Application of intravenous or intrathecal allogenic or autogenic microvesicles or MSC cells may be useful in treating this disorder by lowering inflammation or causing epegentic reprogramming in brain resident immune cells. In one embodiment, the membrane-enclosed vesicle composition is useful for the treatment of autism.

In one embodiment, the membrane-enclosed vesicle composition may be useful for the treatment of infertility. In one embodiment, the membrane-enclosed vesicle composition may be useful for the treatment of autoimmune diseases. In another embodiment, the membrane-enclosed vesicle composition may be useful for the treatment of hair loss. In a certain embodiment, the membrane-enclosed vesicle composition may be useful for the treatment of alopecia areata.

In one embodiment, the membrane-enclosed vesicle composition may be useful for the retention of fat after autologous fat transplant.

In some embodiments, the composition comprising membrane-enclosed vesicle may be an autologous composition. That is, the membrane-enclosed vesicle to be administered to a subject is obtained from said subject or cultured from said subject's cells. In one embodiment, cells from a human subject may be harvested and cultured. The cultured human cells may be induced, stimulated, or engineered to secrete an effective amount of membrane-enclosed vesicle necessary for therapeutic use. In one embodiment, cultured human cells may be induced, stimulated, or engineered to secrete an effective amount of exosomes.

In some embodiments, the composition comprising membrane-enclosed vesicle may be an allogenic composition. That is, the membrane-enclosed vesicle to be administered to a subject is obtained from a different subject, but in the same group of species. For example, in a human subject, the membrane-enclosed vesicle is obtained or cultured from a different individual than those receiving the membrane-enclosed vesicle for therapeutic use.

In some embodiments, the composition comprising membrane-enclosed vesicle may be xenogenic composition. That is, the membrane-enclosed vesicle to be administered to a subject is obtained from an organism of a different species. For example, cells from a donor organism is harvested and cultured to induce, stimulate, or engineer to produce an effective membrane-enclosed vesicle composition.

The membrane-enclosed vesicle composition may be obtained from a variety of cell types. Particularly, one or more cells selected from of the group consisting of mesenchymal stem cell, amnion-derived multipotent progenitor cell, chorion derived mesenchymal stem cell, induced pluripotent stem cell, keratinocyte, fibroblast, embryonic stem cell, ectodermal stromal cell, endodermal stromal cell, olfactory ensheathing cell, dental pulp stem cell, and immortalized mesenchymal stem cell may be useful in harvesting, obtaining, and/or culturing membrane-enclosed vesicle compositions. In one embodiment, the membrane-enclosed vesicle composition is obtained from mesenchymal stem cells.

In one embodiment, the method of culturing the cells for the production of membrane-enclosed vesicles may further involve inducing oxidative stress. The oxidative stress may be induced by an externally added cytokine or by an oxidant such as hydrogen peroxide.

The membrane-enclosed vesicle compositions obtained by any of the process described herein, may be purified and isolated to obtain a composition that is concentrated in particular type of membrane-enclosed vesicle. Alternatively, the membrane-enclosed vesicle composition obtained by any of the process described herein, may be used without separating the different types of membrane-enclosed vesicle contained within.

In one embodiment, the membrane-enclosed vesicle is selected from one or more of endosome, exosome, and microvesicle. In another embodiment, the membrane-enclosed vesicle comprises plasma membrane as the enclosure membrane. In one embodiment, the membrane-enclosed vesicle is derived from the plasma membrane. In a certain embodiment, the plasma membrane-enclosed vesicle is substantially free of major histocompatibility complex (MHC).

In one embodiment, the membrane-enclosed vesicle useful for therapeutic purposes has a diameter range of about 10 nm to about 200 nm. In another embodiment, the diameter range of the membrane-enclosed vesicle is about 30 nm to about 100 nm.

In some embodiments, the cells for producing membrane-enclosed vesicles may be obtained from a human subject. In one embodiment, the cells may be obtained from a human subject who is about 1, 2, 3, 4, 5, 6, 7, 8, 9, 10, 11, or 12 months old. In other embodiments, the cells may be obtained from a human subject who is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 years old. In another embodiment, the cells may be obtained from a human subject who is greater than 50 years old.

### Pharmaceutical Composition Comprising Membrane-Enclosed Vesicle

The membrane-enclosed vesicle composition obtained by various methods disclosed herein, in one embodiment, may be formulated with one or more pharmaceutical acceptable carrier, excipient, adjuvant, diluent and/or binder. Suitable pharmaceutically acceptable carriers, excipients and diluents may include one or more of any and all conventional solvents, dispersion media, fillers, solid carriers, aqueous solutions, coatings, vehicles suitable for topical administration, other antimicrobial agents, isotonic and absorption enhancing or delaying agents, activity enhancing or delaying agents for pharmaceutically active substances, and are well known in the art. Common pharmaceutically acceptable additives are disclosed, by way of example, in Remington: the Science & Practice of Pharmacyby Alfonso Gennaro, 20^{th} ed., Lippencott Williams & Wilkins, (2000). Except insofar as any conventional carrier, excipient or diluent incompatible with the membrane-enclosed vesicle composition, use thereof in the present invention is contemplated.

In one embodiment, suitable pharmaceutically acceptable carriers include, but are not limited to, inert solid fillers or diluents and sterile aqueous or organic solutions (e.g., polyethylene glycol, propylene glycol, polyvinyl pyrrolidone, ethanol, benzyl alcohol, etc.). In certain such embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose, polyvinylpyrrolidone, fillers, such as sugars (e.g., lactose, sucrose, mannitol, or sorbitol), and cellulose preparations (e.g., maize starch, wheat starch, rice starch, potato starch, gelatin, gum tragacanth, methyl cellulose, hydroxypropylmethylcellulose, sodium carboxymethylcellulose, and/or polyvinylpyrrolidone PVP).

In one embodiment, the membrane-enclosed vesicle composition may have one or more externally added additional, compatible, pharmaceutically-active materials. In a certain embodiment, the membrane-enclosed vesicle composition comprises externally added one or more growth factors, nucleic acids, or protein molecules. In some embodiments, the membrane-enclosed vesicle composition comprises one or more growth factors selected from the group consisting of 1,25-dihydroxycholecalciferol, GDF-1, FGF-1, TGF-b, TGF-b2, TGFb3, EGF, miR-133b, bFGF, TIMP1, TIMP2, TIMP3, TIMP4, Wnt4 (protein or mRNA), PDGF-AA, PDGF-BB, G-CSF, VEGF, PGK, MCP-1, IL-6, IL-7, IL-8, IL-10, IDO IL-16, BMP1, BDNF, HGF, KGF, IFNg, MMP-1-9, E-cadherin, Fibronectin, Hsp90, gp96, Myosin, Keratin, Annexin I, aldehyde dehydrogenase, ATP synthase, insulin like growth factor binding protein 1, GM-CSF, IGF like family member, miR-7, miR-100, miR-103, miR-106, miR-107, FOXP3, RANKL, and GDNF.

### Administration of Membrane-Enclosed Vesicle Composition

In some embodiments, the membrane-enclosed vesicle composition of the present disclosure may be administered to a subject by any method known to those of ordinary skill in the art. Non-limiting examples of administration include orally, parenterally, intravenously, nasally, intradermally, intraarterially, intraperitoneally, intralesionally, intracranially, intraarticularly, intraprostaticaly, intraportally, intrapleurally, intratracheally, intrathecally, intravitreally, intravaginally, intrarectally, intratumorally, intramuscularly, subcutaneously, subconjunctival, intravesicularlly, mucosally, intrapericardially, intraumbilically, intraocularally, pulmonary, inhalationally, buccally, sublingually, topically, transdermally, locally, injection, infusion, continuous infusion, localized perfusion bathing target cells directly, via a catheter, via a lavage, directly into a heart chamber, directly injected into the organ or portion of organ or diseased site of interest, or by other method or any combination of the forgoing as would be known to one of ordinary skill in the art. In one embodiment, the membrane-enclosed vesicle composition is administered orally, intravenously, or inhalationally. In another embodiment, the membrane-enclosed vesicle composition is administered in a dosage form selected from the group consisting of solid dosage form, a cream, an aqueous mixture, a lyophilized aqueous mixture and an aerosol.

In one embodiment, the pharmaceutical dosage form comprising the membrane-enclosed vesicles of the present disclosure may include additional pharmaceutically acceptable materials such as dyes, flavoring agents, preservatives, antioxidants, opacifiers, thickening agents and stabilizers. However, these materials, when added, should not unduly interfere with the biological activities of the components of the membrane-enclosed vesicle compositions of the present disclosure.

In one embodiment, the pharmaceutical dosage form comprising the membrane-enclosed vesicle is a liquid (e.g., a suspension, elixir and/or solution). In some embodiments, a liquid pharmaceutical composition is prepared using ingredients known in the art, such as water, glycols, oils, alcohols, flavoring agents, preservatives, and coloring agents.

In one embodiment, the pharmaceutical dosage form comprising the membrane-enclosed vesicle is a solid (e.g., a powder, tablet, and/or capsule). In some embodiments, a solid pharmaceutical composition comprising one or more ingredients known in the art, such as starches, sugars, diluents, granulating agents, lubricants, binders, and disintegrating agents.

In one embodiment, the pharmaceutical dosage form comprising the membrane-enclosed vesicle is formulated as a depot preparation. In some embodiments, depot formulations are administered by implantation (e.g., subcutaneously or intramuscularly) or by intramuscular injection. In some embodiments, depot formulations may comprise suitable polymeric or hydrophobic materials (e.g., an emulsion in an acceptable oil) or ion exchange resins, or as sparingly soluble derivatives, such as, as a sparingly soluble salt.

In one embodiment, the pharmaceutical dosage form comprising the membrane-enclosed vesicle is formulated as a sustained-release system. A non-limiting example a sustained-release formulation is a semi-permeable matrix of solid hydrophobic polymers. In certain embodiments, sustained-release systems may, depending on their chemical nature, release pharmaceutical agents over a period of hours, days, weeks or months.

The membrane-enclosed vesicle composition may have a concentration of membrane-enclosed vesicle that are about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000 ng/ml, μg/ml, mg/ml, or g/ml, or any range derivable therein.

The membrane-enclosed vesicle composition may be administered to or self-administered by the subject 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range derivable therein.

### EXAMPLES

### Example 1

### Cell Growth Study of Fibroblast Cell Monolayer

Mesenchymal stem cells (MSC) have been demonstrated to possess a broad secretome that may impact wound healing. Mesenchymal stem cell secretions have been shown to increase migration of both dermal fibroblasts and epidermal keratinocytes in models of scratch wound healing. MSC supernatant is rich in chemokines such as CCL23, CCL15, CXCL12, CXCL5, CCL2, CXCR3, CCL11, CXCL13, CCL8, CYR61, CCL1. Mesenchymal stem cells aid in tissue regeneration by lowering inflammation and are known to secrete TGF-b and IL-10, initiating and supporting angiogenesis via KDR, VEGF, IL8, ANF, FGF6, and fibroproliferation and epitheliazation in conjunction with SDF-1, EGF, HGF and FGF1. Secreted proteins also include Collagen Type I, Type V, Type VI, XII, and Fibronectin which are integral components of the extracellular matrix. Scar formation is a result of inflammation.

The removal of inflammatory neutrophils and macrophages has been shown to allow full skin repair without or with reduced scarring. The first event in wound repair is inflammation, followed by migration of keratinocytes to the wound. Angiogenesis then takes place and macrophages cause a migration of fibroblasts and differentiation into myofibroblasts, which are contractile and close the wound. These fibroblasts form collagen which cause the bulk of a scar. Therefore, an MSC secretory vesicle treated immune environment which lowers the presence and activation of macrophages and causes an increased trafficking of epidermal keratinocytes to the wound improves the regeneration of normal epidermal tissue while avoiding the disordered deposition of collagen and subsequent hypertrophic scarring.

HACAT Keratinocytes and L929 fibroblasts (Life Technologies) may be used. 24 well tissue culture plates are collagen-coated by incubation with Attachment Factor gelatin solution (Life Technologies) for 2 h at room temperature before rinsing with phosphate buffered saline (PBS, Life Technologies). Each well is seeded with cells (keratinocytes, fibroblasts or both) to a final density of 100,000 cells per well (with co-cultures containing equal numbers of each cell type) and these are maintained at 37° C. and 5% CO2 for 24 h to permit cell adhesion and the formation of a confluent monolayer. These confluent monolayers are then scored with a sterile pipette tip to leave a scratch of approximately 0.4-0.5 mm in width. Culture medium is then immediately removed (along with any dislodged cells). The removed medium is replaced with a fresh serum free culture medium, or with membrane vesicle conditioned medium which has been generated from MSC cultures under serum free conditions (MSC-CM). All scratch assays are performed in quadruplicate.

As demonstrated in Table 1, cells cultured in the presence of membrane vesicle comprising medium (MSC-MV) demonstrate a faster regrowth than cells grown in normal medium.

TABLE 1
Fibroblast cell growth comparison result (% wound closure.)

| | MSC-MV | Control |
|---|---|---|
| 12 h | 12 | 6 |
| 18 h | 50 | 20 |
| 24 h | 100% | 50% |

### Example 2

### Treatment of Second Degree Burns

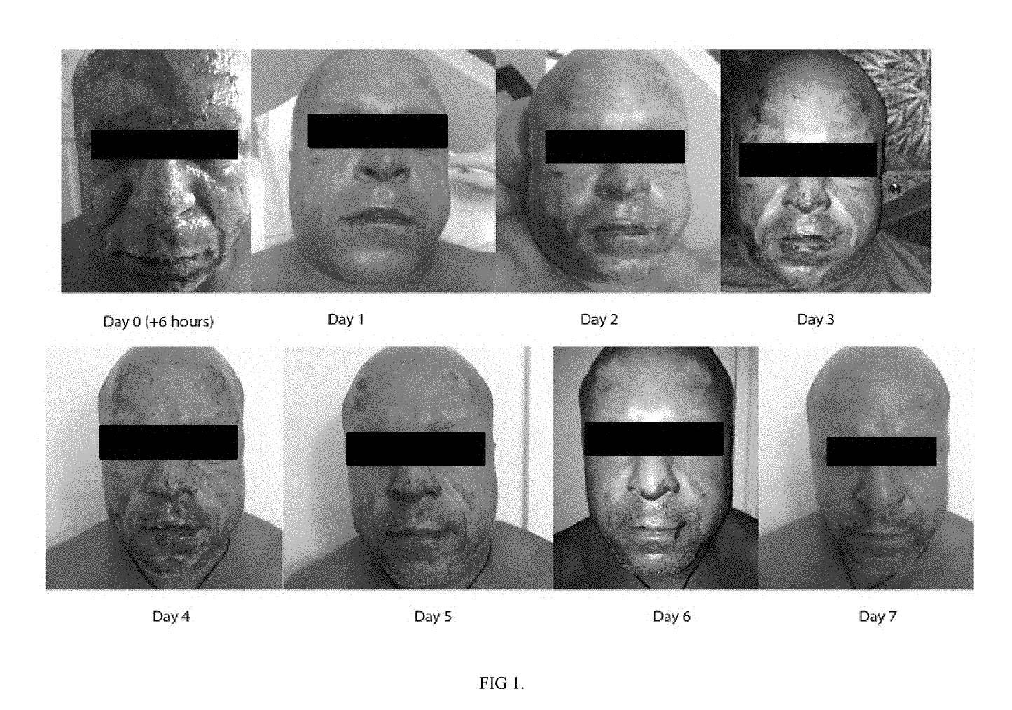

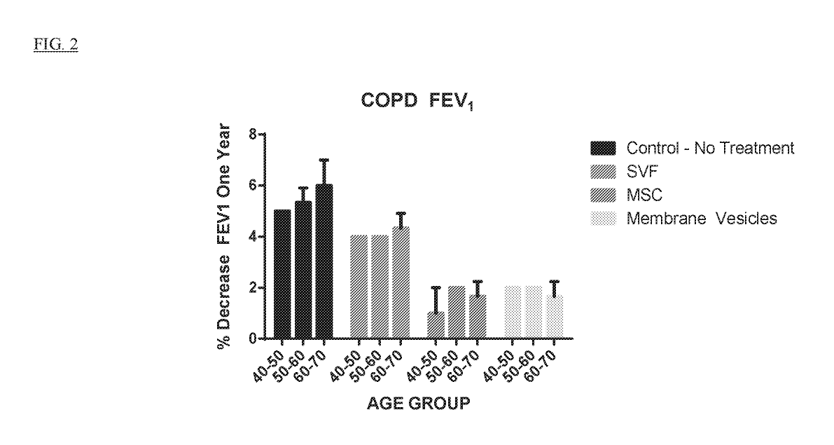

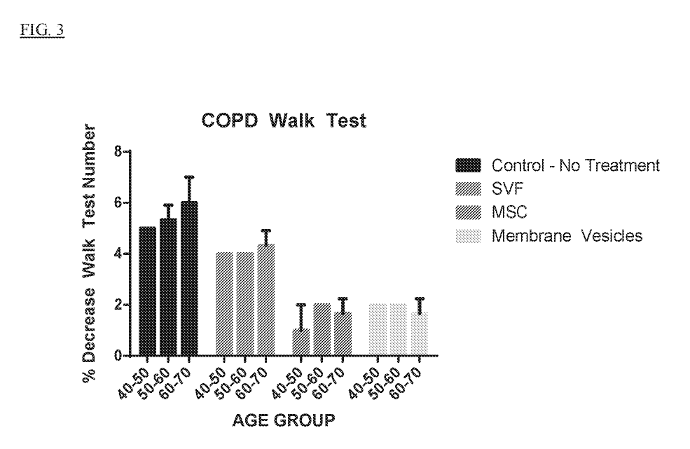

A male patient who suffered a second degree burns to the majority of his face was treated with membrane-enclosed vesicles from allogenic adipose derived mesenchymal stem cells. Adipose derived mesenchymal stem cell membrane vesicles were applied topically to the patient's face using a medium mist spray for a period of 7 days. Patient showed remarkable improvement over the 7 day treatment as illustrated in FIG. 1.

### Example 3

### Treatment of Second Degree Burn with Cream

A study is undertaken to evaluate the effectiveness of the compositions of the present invention in the treatment of patients. The objective of the study is to determine whether application of a cream comprising exosomal vesicles from adipose derived mesenchymal stem cells results in an improvement second degree burns and the prevention of the developments of scars or scar tissues in a patient

A, placebo controlled study is conducted over a 10 day period. A total of 12 subjects (6 men and 6 women, aged 20-55 years), are chosen for the study. An initial assessment of the burns of each patient is made. Three male and three female patients suffering from second degree burns to the body are treated with a cream comprising exosomal vesicles from adipose derived mesenchymal stem exosomal vesicles. As a negative control, three male and three female patients suffering from second degree burns to the body are treated with a cream comprising no exosomal vesicles. The cream is applied topically to the patient's face 12 times a day for a period of 10 days. The severity of the burns is assessed one time a day for up to the ten days of treatment and an initial 7 days after application of the cream. Patients that receive a cream comprising exosomal vesicles begin showing improvement within two days of treatment and remarkable improvement after 7 days. No scarring is visually observed after the 10 day application period or after the 7 day period post-treatment period.

Patients that receive a cream without exosomal vesicles show marginal improvement after 7 days of application of the cream. Significant scarring is visually observed after the 10 day application period and after the 7 day period post-treatment period.

### Example 4

### Treatment of COPD

Patients are included in a placebo controlled double blinded multi-arm trial comparing same day point of service autologous adult adipose derived stem cells to 5 million cultured mesenchymal stem cells to the membrane vesicles present in 500 mls of culture medium exposed to 70-80% confluent mesenchymal stem cells for a 48 hour period. Patients were then assessed for FEV₁ volume and 6 minute walk test ability over a one year period. Data is presented as % decrease in FEV1 (Table 2) and 6 minute walk test (Table 3) results at one year (Control, SVF=Same day liposuction, MSC=5 million MSC.

It should be understood that the above description is only representative of illustrative embodiments and examples. For the convenience of the reader, the above description has focused on a limited number of representative examples of all possible embodiments, examples that teach the principles of the invention. The description has not attempted to exhaustively enumerate all possible variations or even combinations of those variations described. That alternate embodiments may not have been presented for a specific portion of the invention, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the invention. Accordingly, the invention is not intended to be limited to less than the scope set forth in the following claims and equivalents.

### INCORPORATION BY REFERENCE

All references, articles, publications, patents, patent publications, and patent applications cited herein are incorporated by reference in their entireties for all purposes.

However, mention of any reference, article, publication, patent, patent publication, and patent application cited herein is not, and should not be taken as an acknowledgment or any form of suggestion that they constitute valid prior art or form part of the common general knowledge in any country in the world.

We claim:

1. A method of treating and/or reducing the incidence of chronic obstructive pulmonary disease (COPD), comprising administering to a patient in need thereof a therapeutically effective amount of a membrane-enclosed vesicle from a cell.

2. The method of claim 1, wherein the cell is selected from one or more of the group consisting of a mesenchymal stem cell, amnion-derived multipotent progenitor cell, chorion derived mesenchymal stem cell, induced pluripotent stem cell, keratinocyte, fibroblast, embryonic stem cell, ectodermal stromal cell, endodermal stromal cell, olfactory ensheathing cell, dental pulp stem cell, immortalized mesenchymal stem cell.

3. The method of claim 2, wherein the cell is a mesenchymal stem cell.

4. The method of claim 1, wherein the cell is a human cell.

5. The method of claim 1, wherein the membrane-enclosed vesicle is an endosome, an exosome and/or a microvesicle.

6. The method of claim 1, wherein the membrane of the enclosed vesicle is from the plasma membrane.

7. The method of claim 6, wherein the plasma membrane is substantially free of major histocompatibility complex (MHC).

8. The method of claim 1, wherein the membrane-enclosed vesicle is about 10 nanometers to about 200 nanometers in diameter.

9. The method of claim 8, wherein the membrane-enclosed vesicle is about 30 nanometers to about 100 nanometers in diameter.

10. The method of claim 1, wherein the membrane-enclosed vesicle is administered to a patient in one or more dosage forms selected from the group consisting of a solid dosage form, a cream, an aqueous mixture, a lyophilized aqueous mixture and an aerosol.

11. The method of claim 1, wherein the membrane-enclosed vesicle is administered orally, intravenously or by inhalation.

12. The method of claim 1, wherein the membrane membrane-enclosed vesicle is administered in a pharmaceutical composition comprising one or more pharmaceutical acceptable carriers.

13. The method of claim 1, wherein the membrane-enclosed vesicle comprises one or more growth factors selected from the group consisting of GDF-1, FGF-1, TGF-b, TGF-b2, TGFb3, EGF, ILF, miR-133b, bFGF, TIMP1, TIMP2, TIMP3, TIMP4, Wnt4 (protein or mRNA), PDGF-AA, PDGF-BB, G-CSF, VEGF, PGK, MCP-1, IL-6, IL-7, IL-8, IL-10, IDO IL-16, BMP1, BDNF, HGF, KGF, IFNg, MMP-1-9, E-cadherin, Fibronectin, Hsp90, gp96, Myosin, Keratin, Annexin I, Aldehyde Dehydrogenase, ATP synthase, Insulin like growth factor binding protein 1, GM-CSF, IGF like family member, miR-7, miR-100, miR-103, miR-106, miR-107, FOXP3, GDNF.

14. The method of claim 13, wherein at least one of the one or more growth factors are exogenous to the cell.

15. The method of claim 1, wherein the membrane-enclosed vesicle comprises SEQ ID NO: 1.

16. The method of claim 1, wherein the membrane-enclosed vesicle comprises one or more compounds selected from the group consisting of hydrocortisone, bacitracin, neomycin sulfate, Polymyxin B Sulfate, Pramoxine HCL, silver sulfadiazine, calendula, citric acid, and sodium chloride.

17. A method of treating and/or reducing the incidence of chronic obstructive pulmonary disease (COPD), comprising administering to a patient in need thereof a therapeutically effective amount of a membrane-enclosed vesicle from a cell.

18. The method of claim 17, wherein the membrane-enclosed vesicle is about 10 nanometers to about 200 nanometers in diameter.

19. The method of claim 17, wherein the membrane-enclosed vesicle comprises one or more growth factors selected from the group consisting of GDF-1, FGF-1, TGF-b, TGF-b2, TGFb3, EGF, ILF, miR-133b, bFGF, TIMP1, TIMP2, TIMP3, TIMP4, Wnt4 (protein or mRNA), PDGF-AA, PDGF-BB, G-CSF, VEGF, PGK, MCP-1, IL-6, IL-7, IL-8, IL-10, IDO IL-16, BMP1, BDNF, HGF, KGF, IFNg, MMP-1-9, E-cadherin, Fibronectin, Hsp90, gp96, Myosin, Keratin, Annexin I, Aldehyde Dehydrogenase, PSGL-1, ATP synthase, Insulin like growth factor binding protein 1, GM-CSF, IGF like family member, miR-7, miR-100, miR-103, miR-106, miR-107, FOXP3, and GDNF.

20. The method of claim 17, wherein membrane-enclosed vesicle comprises SEQ ID NO: 1.
